(19) United States
(12) Patent Application Publication
(10) Pub. No.: US 2018/0273894 A1
(43) Pub. Date: Sep. 27, 2018

(54) METHOD FOR PREPARING A DEGRADABLE MATERIAL

(71) Applicant: TIANJIN INSTITUTE OF INDUSTRIAL BIOTECHNOLOGY, CHINESE ACADEMY OF SCIENCES, Tianjin (CN)

(72) Inventors: Wenxia Jiang, Tianjin (CN); Ping Yang, Tianjin (CN); Guobing Shao, Tianjin (CN); Qi Liu, Tianjin (CN)

(73) Assignee: TIANJIN INSTITUTE OF INDUSTRIAL BIOTECHNOLOGY, CHINESE ACADEMY OF SCIENCES, Tianjin (CN)

(21) Appl. No.: 15/464,690

(22) Filed: Mar. 21, 2017

(51) Int. Cl. C12N 1/14; C12N 1/22; C08H 8/00

(52) U.S. Cl. CPC C12N 1/14; C08H 8/00; C12N 1/22

(57) ABSTRACT

The disclosure relates to the biotechnology field, and a method for preparing degradable materials, which comprises: inoculating Ceriporia lacerata into a culture medium containing lignocellulose debris for solid-state fermentation. The accession number of the C. lacerata is CGMCC No. 10485. The disclosure also relates to a degradable material obtained by the method and a method for cultivating the C. lacerata. The mycelia of the C. lacerata used herein can enwind and fix the culture medium debris (a lignocellulose material). By dint of the C. lacerata, the lignocellulose material can be prepared into a degradable material without sterilization or anti-bacterial treatment, and the preparation process doesn't require a sterile environment; thus, the preparation cost is decreased remarkably. The preparation process of the (plant-based) degradable material herein is energy saving and environment friendly, and the obtained degradable material is low cost and easy to degrade, and its properties are easy to control.

### FIELD OF THE INVENTION

The present invention relates to the field of biotechnology, in particular to a method for preparing a degradable material, especially by using Ceriporia lacerata.

### BACKGROUND OF THE INVENTION

Foamed plastics are common materials for heat preservation, thermal insulation and cushioning material, and are widely applied in building, packaging, filling and energy absorption fields, etc. However, foamed plastics have two major drawbacks: one is that the raw material is obtained from petroleum; for example, 65 L petroleum is required to produce 1 m³ polystyrene foamed plastics; but petroleum resources are limited, and the degree of dependency on import for petroleum in China has exceeded 60%; therefore, it is imperative to seek for a substitute for petroleum-based foamed plastics; another is that foamed plastics are difficult to decompose in natural environment. Plastic items may cause suffocation and entanglement and disrupt digestion in birds, fish and mammals. Plastic debris not only ruins the beauty of the environment, but also pollutes water resources and soil, endangers livestocks and wild animals, and lays a heavy burden on the ecological environment on the earth. In many countries, a main method for disposing urban wastes is landfilling. On one hand, owing to the fact that foamed plastics are light in weight but large in volume, the selected landfill sites are fully filled by foamed plastics very quickly; consequently, the waste disposal capacity of the landfill sites is severely degraded, and the environment may be polluted severely; on the other hand, after the landfill sites are fully filled, the foundation becomes loose relatively, the harmful substances in the wastes, such as bacteria and viruses, etc., may infiltrate into the soil easily, thus pollute the underground water and endanger the environment; moreover, plastic materials produce dioxin when they are burned; hence, foamed plastic wastes may cause severe secondary pollution to the environment if they are burned. Foamed plastic cushioning materials used for product packages have severely affected product export from developing countries, because foamed plastics are repelled in developed countries.

The research on bio-based degradable plastics has been in the ascendant to solve above problems. However, up to now, all bio-based plastic materials, such as polylactic acid (PLA), poly butylenes succinate (PBS), polypropylene carbonate (PPC), etc., are produced from sugar, which belongs to a food foodstuff of human and animals. Even if lignocellulose is used as the fermentation raw material, lignocellulose has to be hydrolyzed into sugar firstly, then transformed into a monomer for synthesizing plastics through liquid-submerged fermentation or bio-transformation, and finally polymerized into bio-based plastics through a chemical process (PHA is a macromolecular material directly synthesized from sugar through bio-synthesis and accumulated in cells; therefore, raw PHA can be obtained only after a cell wall breaking process). Such processes consume a large amount of raw material and energy, possess complicated process route, complex production technology, high equipment investment, higher costs than that of existing general-purpose plastics, and cause heavy pollution in the production process. Hence, at present, it is difficult to apply such products widely.

Starch plastics, which are relatively matured products, were developed rapidly in the 1980s. The annual growth rate of starch plastics was as high as 75% in USA, and the starch plastics were mainly filled-type starch biodegradable plastics at that time. Starch-based biodegradable plastics are developed most widely up to now, and there are many research entities related with starch-based biodegradable plastics in the world; for example, there are more than 40 research units in China. However, the research results that have been published successively since the 1990s have indicated that only the starch in the starch-based biodegradable plastics can be degraded, while other constituents are broken into fragments and still remain in soil and waters. Therefore, with starch-based biodegradable plastics, the “white pollution” of plastics can't be eliminated by the root.

Terrestrial plants have got cell walls with dense and complex structures mainly formed by cellulose, hemicellulose and lignin in the long-term evolution process. The lignin and hemicellulose constituents are cross-linked with each other and wrap the polysaccharide moiety of fibrin. A mixture of such three-dimensional structures is generally referred to as lignocellulose. Lignocellulose is a renewable biomass in the highest yield. It is considered that lignocellulosic biomass comprises about 50% of world biomass and its annual production was estimated in 10-50 billion ton (Sánchez, ó. J., Cardona, C.A. Trends in Biotechnological Production of Fuel Ethanol from Different Feedstocks. Bioresour. Technol, 2008, 99, 5270-5295). Lignocellulose is an ideal resource for human in the future. At present, up to 6-7 MT renewable lignocellulose resources (e.g., straw wastes) are not exploited and utilized economically and effectively every year in China, and even become a burden on farmers in the countryside. Straw burning still continues despite repeated prohibition, produces a large amount of PM2.5 pollutants and has become a new pollution source in China. How to transform the huge amount of straws into valuable resources has become strategic challenge in the social and economic development in many countries.

If the straw resources can be utilized as a raw material to produce bio-based materials that are widely accepted in the market, attain high economic benefits and are environment friendly with novel fermentation techniques at a low cost without pollution, it will be of great significance for industrial structure adjustment and even for economic and social development.

Though there are some research reports on utilizing microorganisms to prepare bio-based materials presently, the cultivating processes involved in those reports have to be carried out under sterile conditions and have demanding requirements for the cultivating conditions, thus the preparation cost is relatively high.

### SUMMARY OF THE INVENTION

To overcome the drawbacks in the prior art, such as demanding preparation conditions and high production cost, the present invention provides a use of C. lacerata in preparation of a degradable material, in which the degradable material is prepared from low-value lignocellulose debris with a simple method through mycelium growth, enwinding, bonding, and forming.

To attain the object described above, the inventor has selected C. lacerata that can effectively enwind and fix lignocellulose materials through a large quantity of experiments. Accordingly, in a first aspect, the present invention provides a method for preparing a degradable material, comprising: inoculating C. lacerata into a culture medium (or culture substrate) that contains lignocellulose debris for cultivating, wherein, the accession number of the C. lacerata is CGMCC No. 10485.

In a second aspect, the present invention provides a degradable material obtained by the method described in the first aspect. In a third aspect, the present invention provides a method for cultivating C. lacerata, comprising: inoculating C. lacerata deposited as CGMCC No. 10485 into a culture medium for cultivating.

The mycelia of C. lacerata used in the present invention can enwind and fix (or bond) the culture medium debris (lignocellulose debris), and this process doesn't have to be carried out in a sterile environment. By dint of the C. lacerata, a plant material can be used to prepare a degradable material without sterilization or adding any anti-bacterial/bacteriostatic agents, and sterile operation and sterile environment are not required in the preparation process. Thus, the preparation cost is decreased remarkably, and the method can be applied widely. The preparation process of the (plant-based) degradable material in the present invention is energy saving and environment friendly, and the obtained degradable material is low in cost and easy to degrade. Moreover, those skilled in the art can easily design the appearance and the raw material mix ratio according to expected purpose of the material, and thereby obtain a degradable material that meets the requirements in terms of appearance and performance.

Other features and advantages of the present invention will be further detailed in the embodiments hereunder.

### Biological Deposit

The C. lacerata used in the present invention was deposited in China General Microbiological Culture Collection Center (CGMCC) (address: Institute of Microbiology, Chinese Academy of Sciences, Building 3, No. 1 West Beichen Road, Chaoyang District, Beijing, postal code: 100101) on Apr. 28, 2015, and numbered as CGMCC No. 10485.

### BRIEF DESCRIPTION OF THE DRAWINGS

The accompanying drawings are provided here to facilitate further understanding on the present invention, and constitute a part of this document. They are used in conjunction with the following embodiments to explain the present invention, but shall not be comprehended as constituting any limitation to the present invention. In the figures:

FIG. 1 is a flow chart of a preferred embodiment of the method for preparing a degradable material according to the present invention;

FIG. 2 shows the morphology of the C. lacerata used in the present invention after liquid culture, wherein, FIG. 2A shows the morphology of the C. lacerata after cultivating in a sterilized liquid culture medium, while FIG. 2B shows the morphology of the C. lacerata after cultivating in a non-sterilized liquid culture medium;

FIG. 3 shows the morphology of the C. lacerata used in the present invention after solid-state fermentation, wherein, FIG. 3A shows the morphology of a solid culture obtained by inoculating a liquid culture (obtained by cultivating the C. lacerata in a sterilized liquid culture medium) into a solid culture medium and conducting solid-state fermentation therein, while FIG. 3B shows the morphology of a solid culture obtained by inoculating a liquid culture (obtained by cultivating the C. lacerata in a non-sterilized liquid culture medium) into a solid culture medium and conducting solid-state fermentation therein;

FIG. 4 shows the morphologies of degradable materials obtained in two different embodiments of the present invention;

FIG. 5 shows a curve diagram of the change of biomass and pH in the open fermenting process of the C. lacerata used in the present invention in a 20 L fermenter and a 200 L fermenter.

### DETAILED DESCRIPTION OF THE EMBODIMENTS

Hereunder some embodiments of the present invention will be detailed with reference to the accompanying drawings. It should be appreciated that the embodiments described here are only provided to describe and explain the present invention, but shall not be deemed as constituting any limitation to the present invention.

The end points and any value in the ranges disclosed in the present invention are not limited to the exact ranges or values. Instead, those ranges or values shall be comprehended as encompassing values that are close to those ranges or values. For numeric ranges, the end points of the ranges, the end points of the ranges and the discrete point values, and the discrete point values can be combined to obtain one or more new numeric ranges, which shall be deemed as having been disclosed specifically in this document.

The “particle size” involved in the present invention is represented by the mesh size of a material sieve.

The method for preparing a degradable material in the present invention comprises: inoculating C. lacerata into a culture medium that contains lignocellulose debris for cultivating, wherein, the accession number of the C. lacerata is CGMCC No. 10485.

In the present invention, the lignocellulose debris may be obtained from plants, such as at least one of seeds, stalks, roots, leaves, and fruits; namely, the lignocellulose debris may be obtained from wood, cotton, cotton wool, paper, wheat grass, rice straws, sorghum stems, reed, hemp, mulberry bark, Paper mulberry bark, corn stalks, rape straws, Jerusalem artichoke stalks, Chinese pennisetum, couch grass, Chinese silver grass, elephant grass, Pennisetum sinese, rattan, Panicum virgatum, grape vines, sugarcane, energy plants, and their wastes (i.e., plant-based wastes). Alternatively, the lignocellulose debris may be obtained from microorganisms, such as algae (especially medium-size and large-size marine algae, such as kelp and enteromorpha), etc.

As described above, the lignocellulose debris may also be provided from plant-based wastes. The plant-based wastes may be stems and leaves of crops (e.g., straws (including residual stems and leaves of gramineous crops such as paddy rice, wheat, maize, sorghum, etc. after threshing), cotton stems, soybean straws, rape straws, Jerusalem artichoke stalks, Chinese pennisetum, couch grass, Chinese silver grass, elephant grass, Pennisetum sinese, rattan, and Panicum virgatum, etc.), seed hulls (e.g., cotton seed hulls, rice hulls, peanut hulls, wheat bran, and rice bran, etc.), wood wastes (wood flour, offcut, core wood, bark, branches, wrappers, wood shavings, etc.), paper scraps, cotton flocks, maize cobs, and sugar cane bagasse, etc.

More preferably, the plant-based wastes are at least one of soybean straws, corn stalks, wheat bran, cottonseed hulls, peanut hulls, maize cobs and offcuts.

There is no particular restriction on the particle size of the lignocellulose debris; however, to obtain a better forming effect of the degradable material, preferably, the mass of lignocellulose debris in particle size ≥2 mm (more preferably ≤25 mm, even more preferably within a range of 2-15 mm) accounts for 20-100% of the total mass of the lignocellulose debris (e.g., 20%, 30%, 40%, 50%, 60%, 70%, 80%, 90%, 100%, or a range between any two of those values).

In the present invention, the culture medium contains nutrient substances required for growth of the C. lacerata, such as carbon source, nitrogen source, and inorganic salt, etc. There is no particular requirement for the culture medium, as long as the culture medium contains lignocellulose as a carbon source (the amount of the carbon source measured in C may be 0.1-0.65 g carbon source/g culture medium). Preferably, the culture medium may further contain nitrogenous materials as a nitrogen source, such as wheat bran, rice bran, corn steep liquor, yeast extract, soybean meal, protein, hydrolysates of protein, amino acids, urea, ammonia, ammonium salts, nitrates, and nitrites, etc. (the amount of the nitrogen source measured in N may be 2-15 mg nitrogen source/g culture medium). A certain amount of inorganic salts, such as calcium sulfate, may be added into the culture medium as required, and the amount of the inorganic salts may be 5-15 mg inorganic salts/g culture medium.

In the present invention, the lignocellulose debris may also be used as the culture medium directly.

In the present invention, the cultivating may be carried out under conventional C. lacerata cultivating conditions; however, the cultivating conditions preferably include: 15-35° C. and 40-95% of relative humidity (of the cultivating environment). The cultivating time may be selected appropriately according to the inoculum size and the expected purpose of the degradable material; usually, the cultivating time is 5-15 days. The inoculum size of the C. lacerata may be 1-10 g C. lacerata/kg culture medium. It should be noted particularly that the inoculum size and the microorganism content involved in the present invention are measured in dry weight of mycelia (the weight after drying to constant weight at 105° C.); the mass of the culture medium involved in the present invention is also measured in dry weight (the weight after drying to constant weight at 105° C.).

In the present invention, to obtain a degradable material in a predefined shape at preset strength (compression property), the method preferably comprises pre-cultivating, followed by cultivating in a mold, and then optionally cultivating outside of the mold; or cultivating in a mold, optionally followed by cultivating outside of the mold. If the inoculum size of the C. lacerata is 1-10 g C. laceratal/kg culture medium, the method comprises pre-cultivating, followed by cultivating in a mold, and then optionally cultivating outside of the mold; or, if the inoculum size of the C. lacerata is higher than 10 g (>10 g) C. lacerata/kg culture medium and less than or equal to 50 g (≤50 g) C. lacerata/kg culture medium, the method comprises cultivating in a mold, optionally followed by cultivating outside of the mold. The purpose of pre-cultivating is to increase the biomass of mycelia. The main purpose of cultivating in a mold is to fix the culture through mycelium growth and obtain a desired shape. The mold may have any three-dimensional shape (e.g., cubic shape, cuboid shape, or irregular three-dimensional shape). The main purpose of cultivating outside of the mold is to enable the mycelia to further grow on the surface of the culture medium, so as to further improve the strength and appearance of the degradable material.

As described above, if the amount of inoculated C. lacerata is comparatively small (e.g., 1-10 g/kg culture medium) at the beginning of the step of cultivating in a mold, the content of the C. lacerata may be multiplied through the pre-cultivating step. The pre-cultivated material is usually scattered (e.g., by stirring or rubbing) before it is cultivated in a mold. The conditions of pre-cultivating, cultivating in the mold, and cultivating outside of the mold may include 15-35° C. and 40-95% of relative humidity (of the cultivating environment), and may be the same or different from each other.

To attain the above-mentioned purpose of pre-cultivating in a better way, more preferably, in relation to an inoculum size of 1-10 g C. lacerata/kg culture medium, the time of pre-cultivating is 1-5 days (1 day, 2 days, 3 days, 4 days, 5 days, or a range between any two of those values), and optimally is 2-4 days.

To attain the above-mentioned purpose of cultivating in a mold in a better way, more preferably, in relation to an inoculum size of 1-10g C. lacerata/kg culture medium, the time of cultivating in the mold is 1-9 days (1 day, 2 days, 3 days, 4 days, 5 days, 6 days, 7 days, 8 days, 9 days, or a range between any two of those values), and optimally is 3-6 days.

To attain the above-mentioned purpose of cultivating outside of the mold in a better way, more preferably, in relation to an inoculum size of 1-10 g C. lacerata/kg culture medium, the time of cultivating outside of the mold is 1-5 days (1 day, 2 days, 3 days, 4 days, 5 days, or a range between any two of those values), and optimally is 1-3 days.

The C. lacerata used in the present invention has strong contaminating microorganism resistance. Therefore, the cultivation doesn't has to be carried out under sterile conditions; namely, the culture medium used for cultivating doesn't have to be treated by sterilization or anti-bacterial treatment (including various conventional microorganism growth or propagation inhibition measures, such as disinfection and sterilization, etc.), but can be used directly; in addition, after inoculation, the culture medium doesn't have to be isolated aseptically from the external environment for sterile cultivating (the culture medium is a culture medium that is not sterilized or anti-bacterial treated, and/or the cultivating step involved in the method is an open cultivating step (i.e., non-sterile cultivating)), wherein, the sterilization or anti-bacterial treatment includes various sterilization or anti-bacterial treatment measures, such as heat disinfection, thermal sterilization, radio sterilization, microwave sterilization, gas fumigation sterilization, adding antibiotic, adding bactericide and/or bacteriostatic agent and/or antimicrobial agent and/or disinfecting agent and/or lysozyme, etc.

In the present invention, before the C. lacerata is cultivated, the C. lacerata may be activated and seed-cultivated sequentially, wherein, the purpose of activation is to transfer the strain in deposited state into a suitable culture medium for cultivating and recovering the fermenting property; the purpose of seed cultivating is to obtain a large amount of pure and vital mycelia, i.e., obtain C. lacerata with high vitality and enough amount for inoculation. The activation and seed cultivation may be carried out via a conventional method in the art. For example, the activation may comprise: inoculating mycelia of the C. lacerata to a PDA medium, and cultivating for 5-10 days at 20° C. The seed cultivating may comprise: inoculating the activated C. lacerata to a liquid seed culture medium, and cultivating for 3-4 days at 15-35° C. (to obtain more seed liquid, the liquid seed cultivating may be carried out in a way with two or more stages. The PDA medium and the liquid seed culture medium can be selected by those skilled in the art as required, and will not be detailed again. Those skilled in the art should appreciate: though the C. lacerata in the present invention can be used directly to prepare a degradable material without sterilization or anti-bacterial treatment, preferably the passage, storage, and seed cultivating (especially primary seed cultivating) of the C. lacerata are carried out under sterile conditions, in order to obtain a better activation and seed cultivating result.

In the present invention, after solid-state fermentation, the obtained culture may be dried, so as to obtain a finished product of degradable material. The drying method preferably is vacuum drying, hot air drying, steam drying, microwave drying, infrared drying or freeze drying. The drying conditions may be conventional. For example, if hot air drying is used, the drying conditions may include: 50-300° C. (e.g. 50-150° C.) for 0.1-360 h.

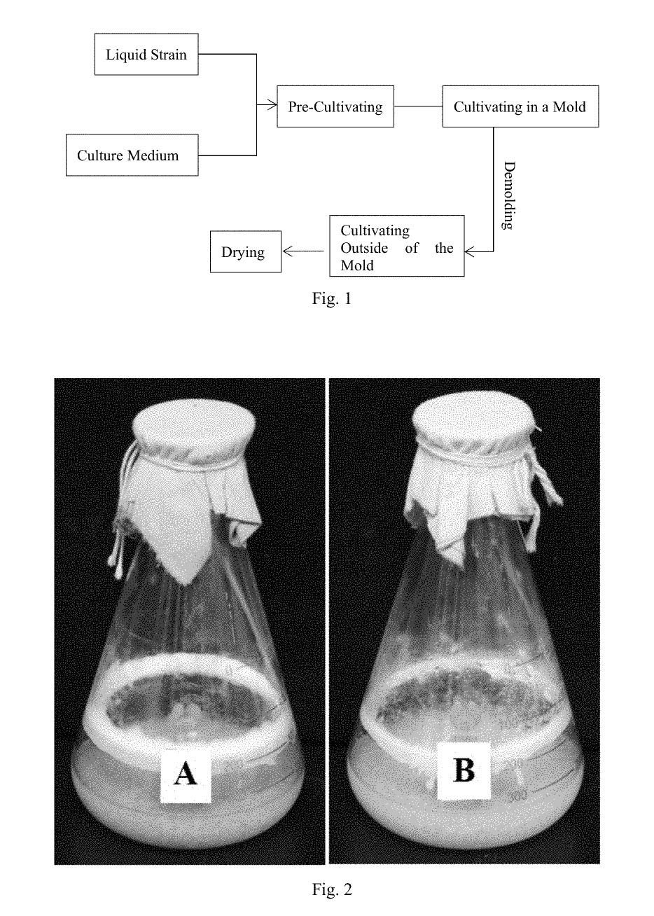

According to a preferred embodiment of the present invention, as shown in FIG. 1, the method for preparing a degradable material in the present invention comprises: inoculating C. lacerata (the accession number of the strain is CGMCC No. 10485) into a culture medium that contains lignocellulose debris, and executing pre-cultivating, cultivating in a mold, and optionally cultivating outside of the mold sequentially, and then drying optionally. The conditions of pre-cultivating, cultivating in the mold, cultivating outside of the mold, and drying have been described above.

The present invention further provides a degradable material prepared by the method described above. The degradable material in the present invention is formed by lignocellulose debris enwound by the C. lacerata, and has a density of 70-400 kg/m³ and a high compression property (e.g., up to 0.5 MPa in a certain embodiment); and is easy to degrade (e.g., the biological decomposition rate (180d) is 75.7% in a certain embodiment); the preparation process is simple; the prepared material is resistant to compression, easy to degrade, light in weight, and has thermal insulating, sound absorbing and shock absorbing properties, and can be used as a substitute for foamed plastics.

The accession number of the C. lacerata used in the present invention is CGMCC No. 10485. The C. lacerata has strong contaminating microorganism resistance, grows rapidly, and can be open-cultivated in a culture medium without sterilization.

The method for cultivating the C. lacerata provided in the present invention comprises: inoculating the C. lacerata into a culture medium for cultivating. The cultivating method may be solid-state fermentation or liquid culture.

Since the C. lacerata used in the present invention has strong contaminating microorganism resistance, preferably the culture medium used for cultivating is a culture medium that is not sterilized or anti-bacterial treated (including various conventional microorganism growth or propagation inhibition measures, such as disinfection and sterilization, etc.), and/or the cultivating is conducted in an open manner (non-sterile cultivating, i.e., the culture medium can be used directly without sterilization or anti-bacterial treatment, and doesn't have to be isolated aseptically from the external environment for sterile cultivating after inoculation; in the case of liquid culture, it is unnecessary to providing sterile air into the culture medium). Wherein, the sterilization or anti-bacterial treatment includes various sterilization or anti-bacterial treatment measures, such as heat disinfection, thermal sterilization, radio sterilization, microwave sterilization, gas fumigation sterilization, adding antibiotic, adding bactericide and/or bacteriostatic agent and/or antimicrobial agent and/or disinfecting agent and/or lysozyme, etc.

In the present invention, there is no particular requirement for the cultivating conditions; in addition, similar to other fungi, the C. lacerata may be cultivated by solid-state fermentation or liquid culture. If the C. lacerata is cultivated by solid-state fermentation, preferably the cultivating conditions include: 15-35° C. and 40-95% of relative humidity (of the cultivating environment). If the C. lacerata is cultivated by liquid culture, the cultivating conditions include: 15-35° C. Moreover, in the case of liquid culture, the content of dissolved oxygen in the cultivating system may be controlled simply by a conventional method (e.g., by introducing compressed air); usually, the saturation of dissolved oxygen is 5-75%.

The cultivating time may be selected appropriately according to the inoculum size and ultimate objective of cultivating; usually, the cultivating time of solid-state fermentation may be 5-15 days. For solid-state fermentation, the inoculum size may be 1-10 g C. lacerata/kg culture medium. In the case of liquid culture, the cultivating time may be 2-5 days.

In the present invention, the culture medium contains nutrient substances required for growth of the C. lacerata, such as carbon source, nitrogen source, and inorganic salt, etc. There is no particular requirement for the culture medium, as long as the culture medium contains a carboniferous material (e.g., glucose, dextrin, cellulose, starch, hemicellulose, or lignin) as a carbon source (for solid-state fermentation, the amount of the carbon source measured in C may be 0.1-0.65 g carbon source/g culture medium; for liquid culture, the amount of the carbon source measured in C in the culture medium may be 20-45 g/L). Preferably, the culture medium may further contain nitrogenous materials as a nitrogen source, such as wheat bran, rice bran, corn steep liquor, yeast extract, soybean meal, protein, hydrolysates of protein, amino acids, urea, ammonia, ammonium salts, nitrates, and nitrites, etc. (for solid-state fermentation, the amount of the nitrogen source measured in N may be 2-15 mg nitrogen source/g culture medium; for liquid culture, the amount of the nitrogen source measured in N in the culture medium may be 0.1-0.55 g/L). In the case of liquid culture, a certain amount of inorganic salts may be added in the culture medium, for example, a phosphate (soluble in water and providing phosphate anions, e.g., selected from at least one of potassium dihydrogen phosphate, dipotassium hydrogen phosphate, sodium hydrogen phosphate, sodium dihydrogen phosphate, di-ammonium phosphate and ammonium dihydrogen phosphate) and calcium sulfate can be added, in an amount of 1-8 mg/L culture medium and 5-15 mg/L culture medium respectively.

In the present invention, the cultivating may be different stages of cultivating C. lacerata, such as activation, seed cultivating, or use (fermentation) stage. Those skilled in the art can easily select the culture mediums and conditions used in different cultivating phases. For example, a PDA culture medium may be used during activation. The conditions of activation may include: 20-35° C. and 5-10 days. A culture medium that contains the following components (measured in mass percentage) may be used during seed cultivating: 1.5-10% soluble starch, 0.5-1% corn steep liquor (measured in dry weight), and 0.1-0.8% potassium dihydrogen phosphate. The conditions of seed cultivating may include: 15-35° C. for 3-4 days (to obtain more seed liquid, the liquid seed cultivating may be carried out in a way with two or more stages).

In the present invention, both liquid culture and solid-state fermentation may be carried out under non-sterile conditions, i.e., any sterile operation is not required. According to a preferred embodiment of the present invention, the C. lacerata cultivating method comprises: inoculating the C. lacerata into a liquid culture medium for seed cultivating, and inoculating the obtained seed liquid into a solid culture medium for solid-state fermentation. The culture medium and conditions have been described above, and will not be detailed again.

Hereunder the present invention will be detailed via examples.

### PREPARATION EXAMPLE 1

C. lacerata (accession number: CGMCC No. 10485, hereafter simply referred to as YY fungus) is inoculated to the slant medium in a Kolle flask, and cultivated in a PDA culture medium at 25° C. for 7 days. The slant culture is inoculated into a liquid culture medium for primary seed, the composition of this culture medium is (in mass percentage): 2% soluble starch, 0.6% dry powder of corn steep liquor, and 0.1% potassium dihydrogen phosphate, natural pH; the culture medium is sterilized at 121° C. for 20 min.; the cultivating conditions include: loaded liquid: 150 mL/500 mL triangular flask, inoculum size: about 3 cm² lawn, and cultivating at 25° C. for 3-4 days with shaking at 150 rpm; thus, a primary seed liquid is obtained.

The primary seed liquid is inoculated into a liquid culture medium for secondary seed, the formulation of this culture medium is (in mass percentage): 6% maize starch, 0.8% dry powder of corn steep liquor, 0.5% potassium dihydrogen phosphate, and 0.0198% alpha-amylase, natural pH; the culture medium is sterilized at 121° C. for 20 min.; the inoculum size is 5% (volume ratio) in relation to the culture medium, the loaded liquid is 150 mL/500 mL triangular flask; the cultivating is carried out at 25° C. for 3-4 days with shaking at 150 r/min. The obtained fermentation broth is used as a seed liquid for solid-state fermentation (dry weight of biomass is 5 g/L).

### EXAMPLE 1

This example is provided to describe the cultural characteristics of the strain involved in the present invention.

(1) The YY fungus is cultivated (at 25° C., for 7 days with shaking at 150 rpm) in a sterilized liquid culture medium for secondary seed and a non-sterilized liquid culture medium for secondary seed (the compositions of the liquid culture media are presented in the Preparation example 1). The YY fungus can grow normally in both liquid culture media, as shown in FIG. 2 (FIG. 2A shows the morphology of the YY fungus cultivated in the sterilized liquid culture medium, while FIG. 2B shows the morphology of the YY fungus cultivated in the non-sterilized liquid culture medium). There is no obvious difference between the morphology of the hypha body cultivated in the sterilized liquid culture medium and the morphology of the hypha body cultivated in the non-sterilized liquid culture medium. Specifically, in both cases, milk white mycelial rings exist above the liquid level, no contaminated sign is observed, and the smells of the fermentation broth are the same to those cultivated under sterilized condition. The results indicate that the YY fungus has characteristics of fast growth and strong contaminating microorganism resistance.

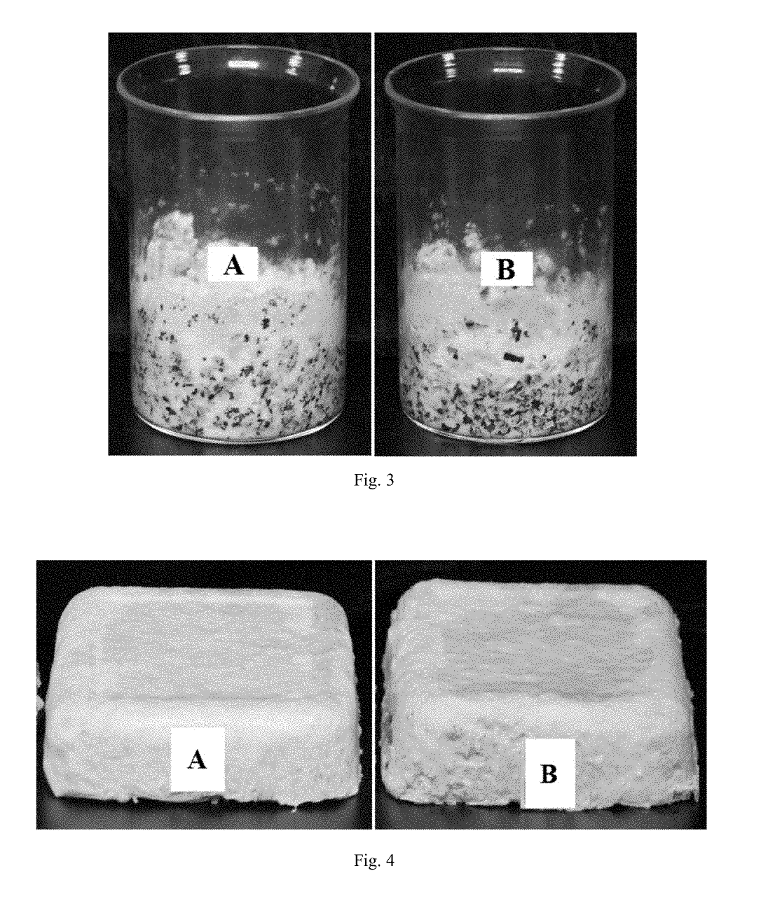

(2) The two fermentation broths (the fermentation broth obtained through cultivating in the sterilized liquid culture medium and the fermentation broth obtained through cultivating in the non-sterilized liquid culture medium in step (1)) are inoculated into a sterilized solid culture medium (the composition is (in mass percentage): 79% cottonseed hulls, 20% wheat bran, and 1% gypsum) and are open-cultivated at 25° C. for 5 days respectively, as shown in FIG. 3 (FIG. 3A shows the morphology of a solid culture (degradable material) obtained by solid-state fermentation the liquid culture (obtained by cultivating the C. lacerata in the sterilized liquid culture medium) in the solid culture medium, while FIG. 3B shows the morphology of a solid culture (degradable material) obtained by solid-state fermentation the liquid culture (obtained by cultivating the C. lacerata in the non-sterilized liquid culture medium) in the solid culture medium). The liquid culture of YY fungus cultivated in the non-sterilized liquid culture medium grows normally after inoculating into the solid culture medium, its growth state doesn't differ from that of the liquid culture of YY fungus cultivated in the sterilized liquid culture medium after inoculating into the solid culture medium.

(3) The YY fungus is open-cultivated in a non-sterilized liquid culture medium (the cultivating conditions are the same as those in step (1), and the difference lies in open cultivating). The YY fungus can grow normally, there is no sign of contamination, and the smell of the fermentation broth is the same as that of the fermentation broth cultivated by shake cultivating in step (1). The fermentation broth obtained by open cultivating is inoculated as a seed into a solid culture medium that is sterilized by steam sterilization (the cultivating conditions as the same as those in step (2). The mycelia sprout and grow normally.

The experimental result indicates that the YY fungus can be liquid-cultivated in a non-sterilized culture medium, and/or the liquid culture can be open-cultivated.

### EXAMPLE 2

This example is provided to describe the method for preparing a degradable material in the present invention.

The seed liquid obtained in Preparation example 1 is mixed with a non-sterilized culture medium (the composition of the culture medium is (in mass percentage): 99% soybean straws (with 0.1-15 mm particle size, the weight ratio of soybean straws in particle size ≤2 mm to soybean straws in particle size >2 mm is 1:1), and 1% gypsum), the inoculum size is 4 g/kg culture medium, open cultivating is carried out at 25° C., and the relative humidity of the cultivating environment is 85%: the seed liquid is pre-cultivated for 3 days, so that the mycelia is fully colonized in the culture medium; after pre-cultivating, the cultivated material is scattered and loaded into a mold, then cultivated for 5 days in the mold; after the mycelia grow fully in the culture medium, the mold is released, and the culture is cultivated for 2 days outside of the mold. Then, the cultivated material is dried at 55° C. for 10h; thus, a degradable material A is obtained, and the morphology of the degradable material A is shown in FIG. 4A.

### EXAMPLE 3

This example is provided to describe the method for preparing a degradable material in the present invention. A degradable material B is prepared by the method used in Example 2, but the particle size of soybean straws is 2 mm or less. The morphology of the obtained degradable material B is shown in FIG. 4B.

### EXAMPLES 4-8

These examples are provided to describe the method for preparing a degradable material in the present invention.

Degradable materials C-G are prepared by the method used in Example 2, but “soybean straws and maize straws mixed at 1:1 mass ratio”, “soybean straws and cottonseed hulls mixed at 1:1 mass ratio”, “soybean straws and maize cobs mixed at 1:1 mass ratio”, “soybean straws and peanut hulls mixed at 1:1 mass ratio”, and “soybean straws and paper scraps mixed at 1:1 mass ratio” are used to replace the “soybean straws”. In all of the cases, the strain can grow normally and the culture shape well.

### EXAMPLE 9

This example is provided to describe the method for preparing a degradable material in the present invention.

The seed liquid obtained in Preparation example 1 is mixed with a non-sterilized culture medium (the composition of the culture medium is (in mass percentage): 99% poplar offcuts (with 2-15 mm particle size), and 1% gypsum), the inoculum size is 10 g/kg culture medium, open cultivating is carried out at 35° C. (the relative humidity of the cultivating environment is 65%): the seed liquid is pre-cultivated for 1 day, so that the mycelia is fully colonized in the culture medium; after pre-cultivating, the cultivated material is scattered and loaded into a mold, then cultivated for 3 days in the mold; after the mycelia grow fully in the culture medium, the mold is released, and the cultivated material is cultivated for 1 day outside of the mold. Then, the cultivated material is dried at 65° C. for 8 h; thus, a degradable material H is obtained, and the shaping effect is essentially the same as that in Example 2.

### EXAMPLE 10

This example is provided to describe the method for preparing a degradable material in the present invention.

The seed liquid obtained in Preparation example 1 is mixed with a non-sterilized culture medium (the composition of the culture medium is (in mass percentage): 99% maize cobs (with 0.1-5 mm particle size, the mass ratio of maize cobs in particle size ≤2 mm to maize cobs in particle size >2 mm is 4:1), and 1% gypsum), the inoculum size is 2 g/kg culture medium, open cultivating is carried out at 15° C. (the relative humidity of the cultivating environment is 55%): the seed liquid is pre-cultivated for 4 days, so that the mycelia is fully colonized in the culture medium; after pre-cultivating, the cultivated material is scattered and loaded into a mold, and cultivated for 8 days in the mold; after the mycelia grow fully in the culture medium, the mold is released, and the culture is cultivated for 3 days outside of the mold. Then, the cultivated material is dried at 80° C. for 20 h; thus, a degradable material I is obtained, and the shaping effect is essentially the same as that in Example 2.

### TESTING EXAMPLE 1

This test example is used to describe the properties of the degradable materials prepared in the present invention.

Through measuring the masses and volumes, the densities of the degradable materials A-I prepared in Examples 2-10 are calculated, respectively;

The degradable materials A-I are tested for compression property (performed in accordance with GB/T 8813-2008 “Rigid Cellular Plastics—Determination of Compression Properties” for the testing method): the compression property is represented by the pressure required for compressing the degradable material to 50%. The higher the required pressure is, the higher the pressure resistance of the degradable material is;

The coefficients of thermal conductivity of the degradable materials A-I are measured with the method defined in GB/T 10294-2008 “Thermal Insulation—Determination of Steady-State Thermal Resistance and Related Properties—Guarded Hot Plate Apparatus”;

The sound absorption coefficients of the degradable materials A-I are measured with the method defined in GB/T 18696.1-2010 “Acoustics—Determination of Sound Absorption Coefficient and Impedance in Impedance Tubes—Part 1: Method Using Standing Wave Ratio”;

The biological decomposition rates (180 d) of the degradable materials A-I are measured by the National Center of Testing and Supervision for Quality of Plastics (NTSQP) with the method defined in GB/T 20197-2006 “Definition, Classification, Marking and Degradability Requirement of Degradable Plastics”;

The measurement results of density, compression property, coefficient of thermal conductivity, sound absorption coefficient, and biological decomposition rate are shown in the following Table 1. The coefficients of thermal conductivity, sound absorption coefficients, and biological decomposition rates of the tested degradable materials are similar, and are not shown fully here.

TABLE 1

| | | | Coefficient | | |
|---|---|---|---|---|---|
| Degradable | | Compression | of Thermal | Sound | Biological |
| Material | Density | Property | Conductivity | Absorption | Decomposition |
| No. | (kg/m³) | (MPa) | (W/(m · K)) | Coefficient | Rate (180 d) |
| A | 159 ± 7 | 0.7 | 0.054 | 0.55 | 75.7% |
| B | 162 ± 16 | 0.025 | — | — | — |
| C | 124 ± 5 | 0.74 | — | — | — |
| D | 235.4 ± 25 | 1.6 | — | — | — |
| E | 199.2 ± 5 | 1.8 | — | — | — |
| F | 199.1 ± 16 | 2.1 | — | — | — |
| G | 166.1 ± 8 | — | — | — | — |
| H | 223.6 ± 9 | 2.3 | — | — | — |
| I | 345.1 ± 23 | 3.5 | — | — | — |

Note:
“—” represents “not tested”.

The degradable materials prepared in the present invention have a high compression property and certain compression strength. According to the measurement results, the degradable materials prepared in the present invention have good thermal insulation and sound absorption properties. In addition, it can be seen from Table 1: the biological decomposition rates (180 d) of the degradable materials prepared in the present invention are higher than the technical requirement (60%) for degradation rate specified in GB/T 20197-2006.

### TESTING EXAMPLE 2

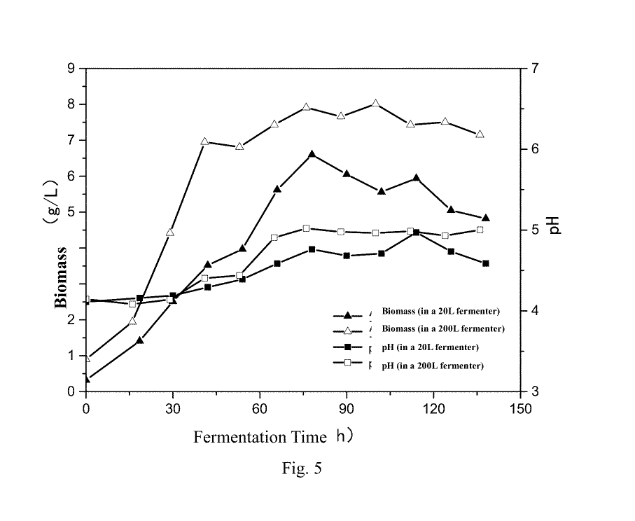

To further verify the properties of the strain in the present invention, the YY fungus is open cultivated in a 20 L fermenter and a 200 L fermenter respectively (the cultivating conditions are the same as those in step (1) in Example 1). In the fermentation process, the top cover of the fermenter is not closed, compressed air is filled into the fermenter without sterile filtration. The change of biomass and the change of pH of the fermentation broth are monitored by sampling during the fermentation process. The result is shown in FIG. 5.

It is verified in open cultivating in the 20 L fermenter and 200 L fermenter: the YY fungus grows normally, no contaminating microorganism grows therein, the smell of the fermentation broth is the same as that in sterile cultivating, the pH change is within a range of 4-5, and the biomass reaches a peak approximately at 75 h (6.6g/L and 8.0 g/L respectively), and the biomass is close to that in sterile shake cultivating. The result further indicates that the YY fungus has characteristics of fast growth and strong contaminating microorganism resistance, etc.

While some preferred embodiments of the present invention are described above, the present invention is not limited to the details in those embodiments. Those skilled in the art can make modifications and variations to the technical scheme of the present invention, without departing from the spirit of the present invention. However, all these modifications and variations shall be deemed as falling into the protected scope of the present invention.

In addition, it should be noted that the specific technical features described in above embodiments can be combined in any appropriate form, provided that there is no conflict. To avoid unnecessary repetition, the possible combinations are not described specifically in the present invention.

Moreover, different embodiments of the present invention can be combined freely as required, as long as the combinations don't deviate from the idea and spirit of the present invention. However, such combinations shall also be deemed as falling into the scope disclosed in the present invention.

1. A method for preparing a degradable material, which is formed by lignocellulose debris enwound by Ceriporia lacerata, the method comprising: inoculating Ceriporia lacerata into a culture medium that contains lignocellulose debris, conducting solid-state fermentation under non-sterile conditions in the culture medium, and thereby enwinding the lignocellulose debris with the Ceriporia lacerata and forming the degradable material, wherein the accession number of the Ceriporia lacerata is CGMCC No. 10485.

2. The method according to claim 1, wherein the weight of lignocellulose debris in particle size ≤2 mm accounts for 20-100% of the total weight of lignocellulose debris; or, the weight of lignocellulose debris in 2-25 mm particle size accounts for 20-100% of the total weight of lignocellulose debris.

3. The method according to claim 1, wherein the solid-state fermentation conditions include: 15-35° C. and 40-95% of relative humidity.

4. The method according to claim 1, wherein if the inoculum size of the C. lacerata is 1-10 g C. lacerata/kg culture medium, the method comprises: pre-cultivating, followed by cultivating in a mold, and then optionally cultivating outside of the mold; or, if the inoculum size of the C. lacerata is higher than 10 g C. lacerata/kg culture medium and less than or equal to 50 g C. lacerata/kg culture medium, the method comprises: cultivating in a mold, optionally followed by cultivating outside of the mold.

5. The method according to claim 4, wherein in relation to an inoculum size of 1-10 g C. lacerata/kg culture medium, the time of pre-cultivating is 1-5 days.

6. The method according to claim 4, wherein in relation to an inoculum size of 1-10 g C. lacerata/kg culture medium, the time of cultivating in the mold is 1-9 days.

7. The method according to claim 4, wherein in relation to an inoculum size of 1-10 g C. lacerata/kg culture medium, the time of cultivating outside of the mold is 1-5 days.

8. The method according to claim 1, wherein the culture medium is a culture medium that is not sterilized or anti-bacterial treated, and/or the culture medium is not isolated aseptically from the external environment.

9. The method according to claim 1, further comprising: drying the culture obtained through solid-state fermentation, wherein the drying method is vacuum drying, hot air drying, steam drying, microwave drying, infrared drying, or freeze drying.

10. A degradable material obtained by the method according to claim 1.

11. A method for cultivating C. lacerata, comprising: inoculating C. lacerata with accession number CGMCC No. 10485 into a culture medium for cultivating.

12. The method according to claim 11, wherein the culture medium is a culture medium that is not sterilized or anti-bacterial treated.

13. The method according to claim 11, wherein the cultivating is conducted in an open manner.

14. The method according to claim 11, wherein the cultivating is solid-state fermentation, and the cultivating conditions include: 15-35° C. and 40-95% of relative humidity.

15. The method according to claim 11, wherein the cultivating is liquid culture, and the cultivating conditions include: 15-35° C.

16. The method according to claim 11, comprising: inoculating the C. lacerata into a liquid culture medium for cultivating a seed liquid, and then inoculating the obtained seed liquid into a solid culture medium for solid-state fermentation.

17. The method of claim 1, wherein the C. lacerata is seed-cultivated in an open manner prior to the step of inoculation into a culture medium that contains lignocellulose debris.

18. The method of claim 17, wherein the medium used in seed-cultivation is a medium that is not sterilized or anti-bacterial treated.
